(19) United States
(12) Patent Application Publication
(10) Pub. No.: US 2012/0191215 A1
(43) Pub. Date: Jul. 26, 2012

(54) HIATAL HERNIA REDUCTION DEVICE AND METHOD

(75) Inventors: Randal S. Baker, Ada, MI (US); Frederick J. Walburn, Grand Rapids, MI (US)

(73) Assignee: Sentinel Group, LLC, Grand Rapids, MI (US)

(21) Appl. No.: 13/189,086

(22) Filed: Jul. 22, 2011

Related U.S. Application Data
(63) Continuation of application No. 13/060,234, filed on May 6, 2011.
(60) Provisional application No. 61/090,750, filed on Aug. 21, 2008.

(51) Int. Cl. A61F 2/04

(52) U.S. Cl. 623/23.64

(57) ABSTRACT

A hiatal hernia reduction device and method includes positioning a device having a wall that is generally configured to the normal anatomy at the gastro-esophageal region of a person without a hiatal hernia. The anatomy of the patient at the gastro-esophageal region is formed substantially to the anatomy of the normal person without a hiatal hernia.

### CROSS REFERENCE TO RELATED APPLICATIONS

The present invention is a continuation of U.S. patent application Ser. No. 13/060,234, filed on Feb. 22, 2011, which is a 371 of International Patent Application No. PCT/US2009/004781, filed on Aug. 21, 2009, which claims priority from U.S. provisional patent application Ser. No. 61/090,750, filed on Aug. 21, 2008, the disclosures of which are hereby incorporated herein by reference in their entirety.

### BACKGROUND OF THE INVENTION

The present invention is directed to a gastro-esophageal device and method.

Hiatal hernia is a result of a protrusion of the stomach upward into the chest cavity through the esophageal hiatus of the diaphragm. Obesity may increase intra-abdominal pressure thereby contributing to hernia formation. It is quite common for GERD and hiatal hernia to be present together in a patient. There are various procedures for treating GERD and/or for treating a hiatal hernia.

### SUMMARY OF THE INVENTION

An aspect of the present invention is directed to a method and device for reducing hiatal hernia.

A hiatal hernia reduction device and method of reducing hiatal hernia, according to an aspect of the invention, includes providing a wall that is generally configured to the normal anatomy at the gastro-esophageal region of a person without a hiatal hernia. The device is positioned at the gastro-esophageal region of the patient. The anatomy of the patient at the gastro-esophageal region is formed to the anatomy of the normal person without a hiatal hernia. The wall may apply an outward force at the gastro-esophageal region to form the anatomy at the anatomy of the patient.

The device may include a cardiac member having a surface that is generally configured to the normal anatomy at the cardiac region of a person without a hiatal hernia and the surface positioned against the cardiac portion of the stomach to restore the shape of the cardiac portion of the stomach to the shape of the cardiac portion of the stomach of the person without a hiatal hernia. The device may include an esophageal member that is generally configured to the normal anatomy at the distal esophagus of a person without a hiatal hernia; with the surface positioned within the distal portion of the esophagus adjacent the gastro-esophageal (GE) junction to restore the shape of the distal esophagus to the shape of the distal esophagus of a person without GERD. The device may further include a connector connected with the esophageal member and the cardiac member to establish intra-abdominal relationship of the distal esophagus, the GE junction and the cardiac region of a person without a hiatal hernia. The connector may be positioned at the GE junction with a contiguous portion of the GE junction substantially unrestrained. At least a portion of the connector may be positioned within the pseudo-sphincter comprising the GE junction. At least a portion of the connector may be positioned outside of the pseudo-sphincter comprising the GE junction.

The angle of HIS of the patient may be affected by positioning the cardiac member with respect to said esophageal member using the connector. Such positioning may include drawing the cardiac member and the esophageal member toward each other.

These and other objects, advantages and features of this invention will become apparent upon review of the following specification in conjunction with the drawings.

### BRIEF DESCRIPTION OF THE DRAWINGS

FIG. 1 is an elevation of a gastro-esophageal device deployed in a patient as viewed from the anterior of a frontal plane;

FIG. 2 is an illustration of a patient's gastro-esophageal (GE) junction as viewed from a transverse plane in a dilated state with a gastro-esophageal device in place;

FIG. 3 is the same view as FIG. 2 with the GE junction in a constricted state;

FIG. 4 is a perspective view of an alternative embodiment of the gastro-esophageal device in FIG. 1 as viewed generally from the posterior of a frontal plane as placed in a patient;

FIG. 5 is a perspective view of the embodiment of the gastro-esophageal device in FIG. 1 as viewed from the direction of the angle of HIS;

FIG. 6 is an elevation of the embodiment of the gastro-esophageal device in FIG. 1 as viewed generally from the posterior of a frontal plane as placed in a patient;

FIG. 7 is a chart of a method for reducing gastro-esophageal reflux disease;

FIG. 8 is a chart of a method for reducing a hiatal hernia; and

FIG. 9 is a chart of a method for reducing stress in a patient.

### DESCRIPTION OF THE PREFERRED EMBODIMENT

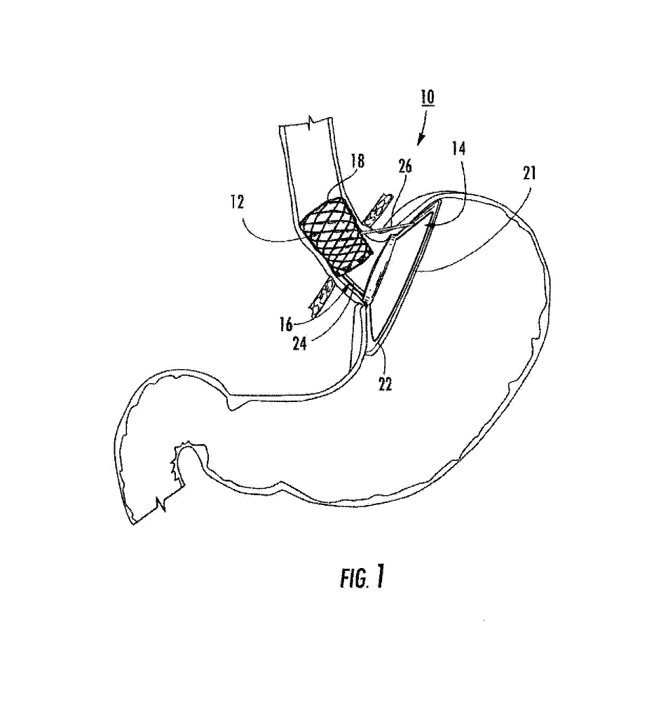

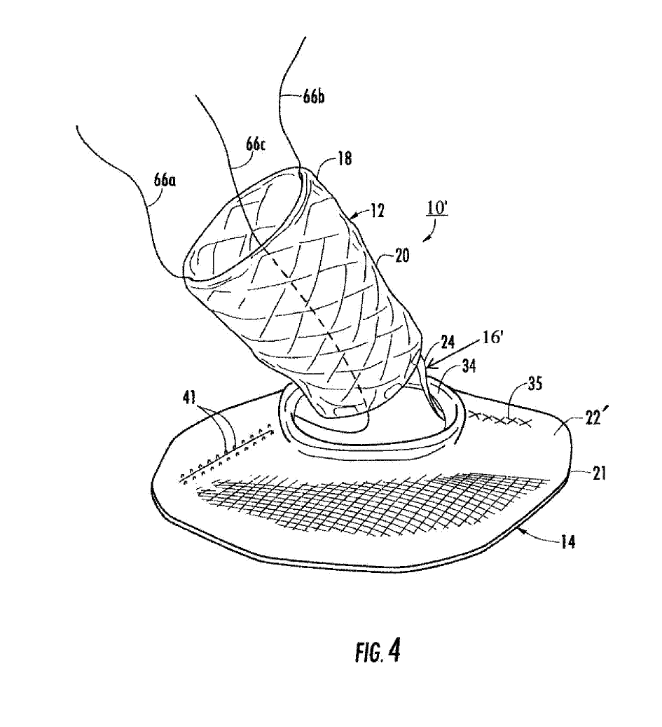

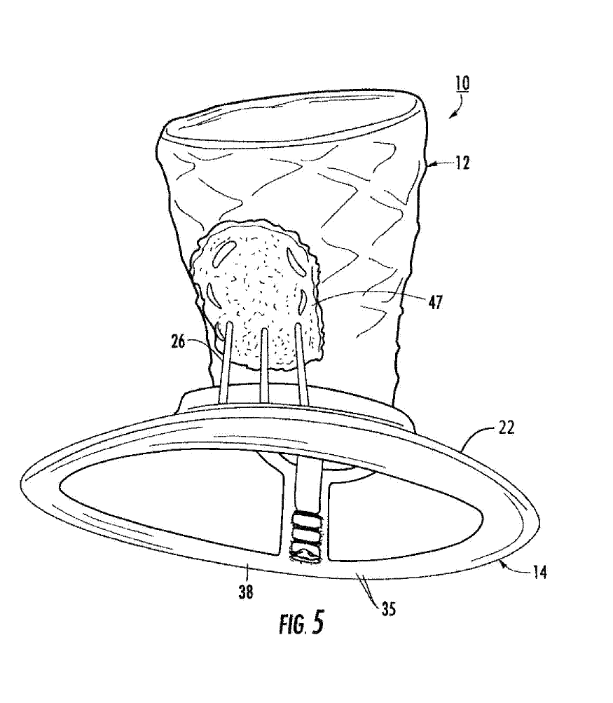

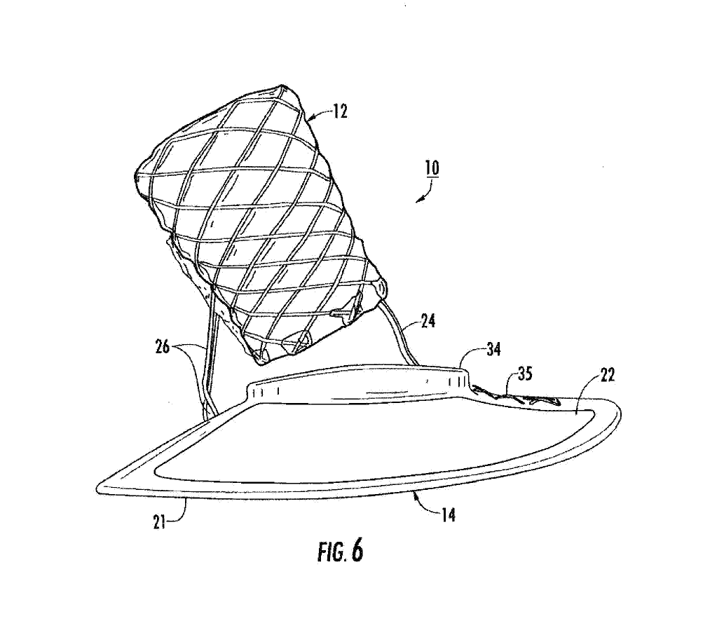

Referring now specifically to the drawings, and the illustrative embodiments depicted therein, a gastro-esophageal device 10 includes an esophageal member 12, a cardiac member 14, and a connector 16 that is connected with esophageal member 12 and cardiac member 14 (FIGS. 1, 5 and 6). Esophageal member 12 has a wall 18 defining an esophageal surface 20 that is configured to generally conform to the shape and size of a portion of the esophagus, namely, at the abdominal portion of the esophagus adjacent the gastro-esophageal (GE) junction. While illustrated as cylindrical in shape, wall 18 may be other shapes, such as a portion of a cylinder, or the like. Cardiac member 14 includes a body 21 defining a cardiac surface that is configured to generally conform to the shape and size of at least a portion of the cardiac portion of the stomach. The cardiac surface may be hoop-shaped cardiac surface 22, as illustrated as FIGS. 1 and 5, or may be a cardiac surface 22′ in the form of a disk, as illustrated in FIG. 4, or a combination of both. Other configurations of body 21 that conform to the shape and size of at least a portion of the cardiac portion of the stomach will be apparent to the skilled artisan. For example, body 21 may take the form of the various embodiments disclosed in International Patent Application Publication No. WO 2008/101048 and U.S. patent application Publication No. 2010/0030017 A1, the disclosures of which are hereby incorporated herein by reference. Connector 16, which is illustrated as a system of connector members, joins the esophageal and cardiac members. Connector 16 may include a tension member 24, which may be a semi-rigid strap which passes through the GE junction.

Connector 16 may include one or more tethers 26, which may be attached to the esophageal and cardiac members and pass from the esophageal member to the cardiac member outside of the GE junction. As will be described in more detail below, this is accomplished by passing tether 26 through wall 18 of esophageal member 12 through the wall of the esophagus, through the wall of the stomach at the cardiac region, and through body 21 of the cardiac member. In an alternative embodiment illustrated in FIG. 4, a gastro-esophageal device 10′ is provided having a connector 16′ that includes a tension member 24, but does not have a tether. The esophageal member 12 and cardiac member 14 of gastro-esophageal 10′ are joined by tension member 24. Other embodiments will be apparent to the skilled artisan. For example, it may be possible to provide a gastro-esophageal device having a cardiac member without an esophageal member provided that an anchor mechanism is provided to anchor the cardiac member to the cardiac region of the stomach.

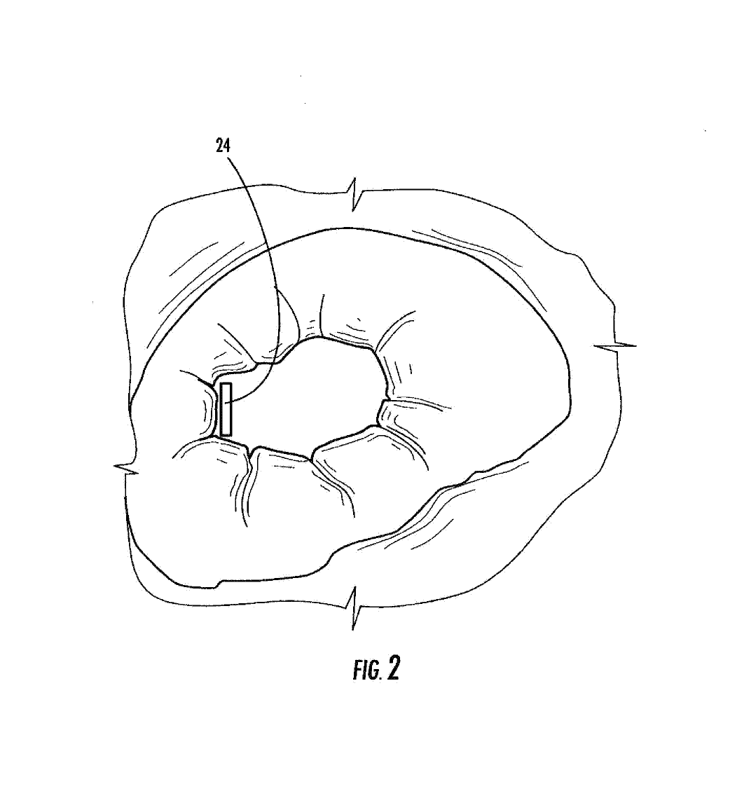

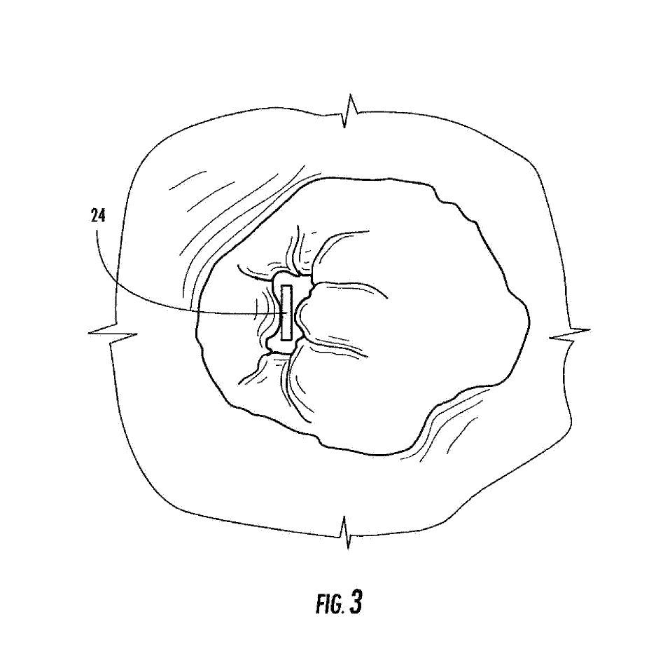

In the illustrated embodiments, connector 16, 16′ leaves most of the GE junction unrestrained. As best seen by comparing FIGS. 2 and 3, tension member 24 may be positioned opposite the angle of HIS. The portion of the pseudo-sphincter of the GE junction at the angle of HIS is relatively unrestrained by the connector and can constrict against the remainder of the pseudo-sphincter and tension member 24, as best seen in FIG. 3. Tension member 24 may include an inwardly curved portion 28 that allows the portion of the GE junction pseudo-sphincter opposite the angle of HIS to assume a relatively normal posture, again as illustrated in FIGS. 2 and 3. As best illustrated in FIG. 1, tether(s) 26 passes outside of the GE junction pseudo-sphincter through the walls of the esophagus and stomach. Once again, the pseudo-sphincter of the GE junction is allowed to constrict in a relatively unrestrained manner because the tether(s) do not significantly interfere with the pseudo-sphincter of the GE junction. Thus, connector 16, 16′ allows the GE junction to dilate for the passage of food (FIG. 2) and constrict when food is not passing (FIG. 3) in order to resist passage of stomach contents into the esophagus. Also, the GE junction pseudo-sphincter is allowed to function in a relatively normal manner for the purposes of belching, vomiting, and the like.

In the illustrated embodiment, most of the GE junction pseudo-sphincter is allowed to operate without substantial restraint. Indeed, at least 75 percent, and even 90 percent, of the GE junction may be unrestrained in order to function in a relatively normal fashion. In one embodiment, tension member 24 is made of a 0.014 inch super-elastic Nitinol sheet. Because the tension member is in the form of a relatively thin semi-flexible strap in this embodiment, it is able to be folded back for the purpose of inserting the gastro-esophageal device through the esophagus for deployment, in a manner that will be described in more detail below. In an alternative embodiment, tension member 24 may be made from a more flexible material, such as Nitinol wire or ePTFE fiber marketed by Gore & Associates, Inc. Tether(s) 26 may be an elongated filament, such as an uncoated silk suture, an Ethibond suture, an ePTFE suture, an elastic line, or the like. If desired, tether(s) 26 may be within a sheath to allow the filament to move lengthwise, for example, to facilitate subsequent adjustment of the spacing between the esophageal and cardiac members to adjust the degree of satiety. An uncoated silk suture may produce fibrous tissue, which may prevent lateral drift of the tether through the tissue at the GE junction. Alternatively, a tether 126 may be used having a surface that promotes tissue attachment and/or tissue ingrowth to prevent lateral drift of the tether.

Gastro-esophageal device 10, 10′ may define a gastro-esophageal reflux disease (GERD) reduction device. Device 10, 10′ has a wall 18, 21 that is generally configured to the anatomy at the gastro-esophageal (GE) region of a person with normal anatomy at the GE junction. For the purposes of discussion herein, a person with a normal anatomy at the GE junction is a person that does not have reflux at the GE junction. It is possible to have reflux, but not to have GERD. Gastro-esophageal device 10, 10′ has a size and configuration to form the anatomy of the patient at the gastro-esophageal region to the anatomy of the normal person without reflux. Thus, esophageal member 12 helps to maintain a generally circular cross section of the distal portion of the esophagus and normal positioning of the distal portion of the esophagus which helps to optimize the proper functioning of the pseudo-sphincter at the gastro-esophageal junction. Also, cardiac member 14 helps to maintain a generally dome shape of the cardiac region of the stomach adjacent the GE junction, which also helps to optimize the proper functioning of the pseudo-sphincter at the GE junction. Because esophageal member 12 and cardiac member 14 are held in a particular relationship by tension member 24 and/or tether(s) 26, device 10, 10′ tends to maintain proper intra-abdominal relationship for the distal esophagus, the gastro-esophageal junction and the upper cardiac portion of the stomach. Device 10, 10′ may also help to affect the angle of HIS by squeezing the tissue so that the infolding of the angle of HIS is able to properly oppose the opposite side of the GE junction, which also helps to optimize the proper functioning of the pseudo-sphincter. Thus, by putting pressure on the wall of the patient in the GE region, device 10, 10′ causes the angle of HIS region and the cardiac region to conform to a more normal anatomy that allows optimal functioning of the pseudo-sphincter of the GE junction. This may be further enhanced by operation of the esophageal member providing a more normal anatomy at the distal esophagus that further assists in optimal functioning of the pseudo-sphincter at the GE junction.

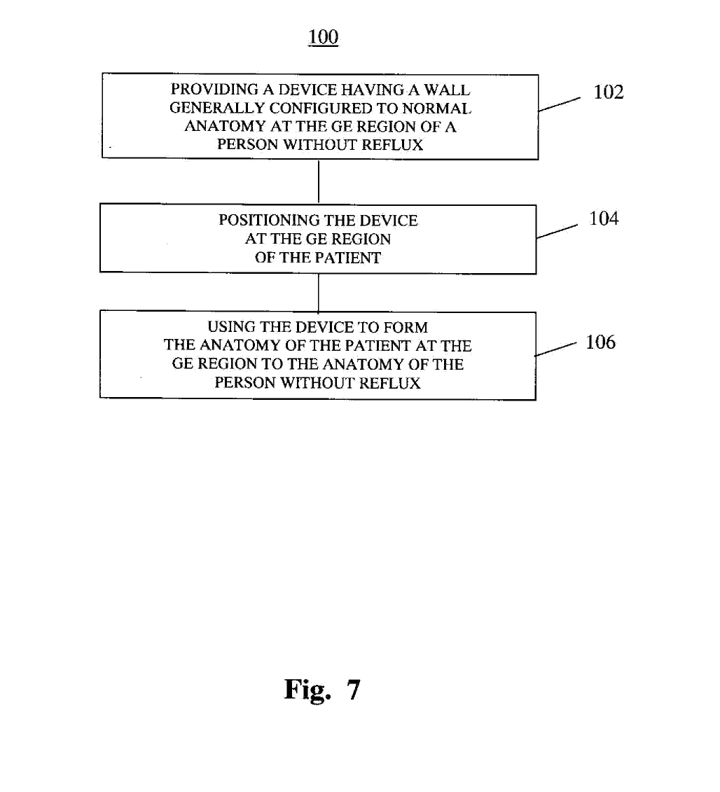

In a method 100 for reducing gastro-esophageal reflux disease in a patient, device 10, 10′, having a wall generally configured to the normal anatomy of the GE region of a person without reflux (102), is positioned at the GE region of the patient at 104 (FIG. 7). Device 10, 10′ is used to form the anatomy of the patient at the GE region to the anatomy of the person without reflux (106).

Gastro-esophageal device 10, 10′ may define a hiatal hernia reduction device. Cardiac member 14 helps to maintain a generally dome shape of the cardiac region of the stomach adjacent the GE junction similar to the anatomy of a person without a hiatal hernia. Also, it helps to maintain the proper intra-abdominal relationship of the distal esophagus, the GE junction and the cardiac region of the stomach. This relationship tends to pull the protrusion of the stomach downwardly from the chest and below the esophageal hiatus of the diaphragm to the abdomen and to reduce slippage of the stomach up through the hiatal defects in order to reduce the hiatal hernia. Also, by providing broad support for the cardiac region of the stomach, the cardiac member resists the stomach being pushed through the hiatus at the diaphragm. In addition to reducing a hiatal hernia, the ability of gastro-esophageal device 10, 10′ to maintain a more normal intra-abdominal relationship of the distal esophagus, the GE junction and the cardiac region of the stomach also serve to reduce the tendency of a hiatal hernia to recur in the future. Thus, gastro-esophageal device 10, 10′ is capable of both reducing a hiatal hernia and resisting its recurrence.

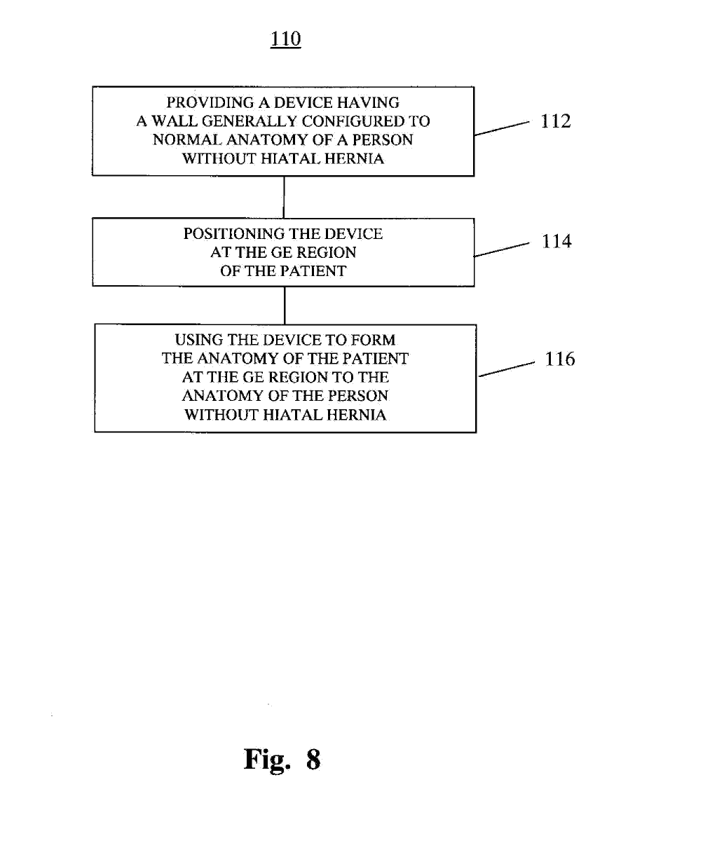

A method 110 for reducing a hiatal hernia in a patient includes positioning the gastro-esophageal device 10, 10′, having a wall generally configured to the normal anatomy of a person without a hiatal hernia (112), at the GE gastro-esophageal region of the patient at 114 (FIG. 8). Device 10, 10′ is used to form the anatomy of the patient at the GE region to the anatomy of the person without the hiatal hernia, as described above.

Gastro-esophageal device 10, 10′ may define a stress reduction device in a patient. Because stress has been generally linked to depression, gastro-esophageal device 10, 10′ may define an anti-depression device. This is accomplished by device 10, 10′ having a wall 18, 21 and applying a strain, such as an outward force with the wall at the gastro-esophageal region, thereby increasing generation of at least one neuro-humoral transmitter. An example of a neuro-humoral transmitter is a neurotransmitter, such as serotonin, norepinephrine, or the like. Another example of a neuro-humoral transmitter is a humoral transmitter, such as ghrelin, leptin, endorphin, or the like. Such humoral transmitter may be locally or distally effective.

As will be described in more detail below, connector 16 may be generally in tension and cardiac surface 22, 22′ stimulates mechanoreceptors in the cardiac region of the patient. While the precise manner of causing this effect is not completely known, it is known that some people often eat to receive emotional, as well as hunger-suppressing, effects. This may be accomplished by the release of neuro-humoral transmitters as a result of the stimulation of mechanoreceptors in the stomach. Because such receptors are dense in the cardiac region, the strain applied by wall 21 on the cardiac region will stimulate these mechanoreceptors. It is believed that this will result in the release of neuro-humoral transmitters. Such transmitters act on the hippocampus, which is a key location for stress regulation in the brain. As previously set forth, stress is known to be linked to depression. Esophageal surface 20 may additionally contribute to release of neuro-humoral transmitters in addition to its function to work in collaboration with connector 16 and cardiac member 14 to stimulate the mechanoreceptors in the cardiac region of the stomach. Also, connector 16, 16′, particularly tension member 24, may apply pressure at a portion of the GE junction and, thereby, assist in generating neuro-humoral transmitters.

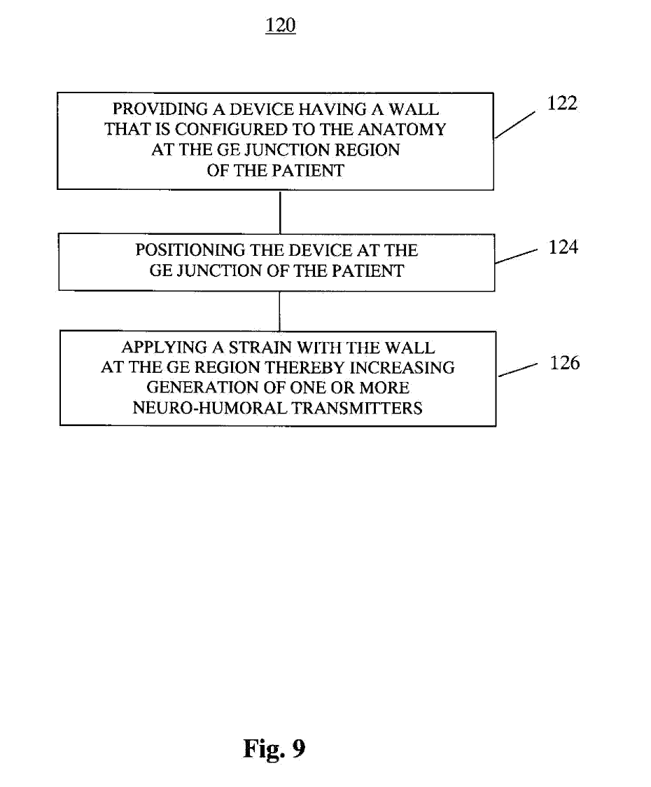

A method 124, which reduces stress in a patient, includes providing device 10, 10′ having a wall configured to the anatomy at the GE region of the patient (122) and positioning device 10, 10′ at the GE region of the patient at 124 (FIG. 9). Walls 18 and/or 21 apply a strain at the GE region thereby increasing generation of one or more neuro-humoral transmitters at 126. As previously set forth, the reduction of stress has a tendency to reduce depression.

As previously described, cardiac surface 22, 22′ of cardiac member 14 and the esophageal surface of the esophageal member are configured to stimulate mechanoreceptors at the abdominal portion of the esophagus, the esophageal-gastric junction and/or the cardia of the patient. The mechanoreceptors stimulated may be tension receptors, which are sensitive to contraction and elongation; stretch receptors, which are sensitive to elongation only; and/or baroreceptors, which are stimulated by change in pressure. This stimulation may be accomplished by cardiac surface 22 and esophageal surface 20 exerting a strain, such as an outward pressure, typically a generally radial outward pressure, to the wall of the cardiac region of the stomach and the abdominal portion of the esophagus. This may be accomplished, at least in part, by the connector 16 transmitting forces between the esophageal member and the cardiac member to press cardiac surface 22 against the cardia. It may also be accomplished, at least in part, by configuring the wall of the esophageal member to create an interference fit with the abdominal portion of the esophagus. The gastro-esophageal device may, alternatively, apply an inward force on the abdominal portion of the abdominal portion of the esophagus, the gastro-esophageal junction and/or cardia. The gastro-esophageal device may, alternatively, apply a longitudinal force, such as a proximal/distal force, to the abdominal portion of the esophagus, the esophageal-gastric junction and/or the cardia.

The strain exerted by the gastro-esophageal device at the abdominal portion of the esophagus, the esophageal-gastric junction and/or the cardiac portion of the stomach is intended to be relatively consistent over as large an area as reasonably possible.

Tether(s) 26 serves to resist distal migration because the tether passes through the esophageal wall and the stomach wall and creates a sort of sandwiching of the esophageal wall and the stomach wall between esophageal surface 20 and cardiac surface 22. This is due, in part, to the upward extension of the cardia at the angle of HIS to be somewhat parallel to the esophageal wall. Connector 16 also serves to bring cardiac surface 22 into engagement with the cardia in order to stimulate the neuroreceptors, which are dominant in the cardia. It also helps to maintain the proper intra-abdominal relationship of the distal esophagus, the GE junction, and the cardiac region to that of a normal person. Thus, it is seen that esophageal member 12, cardiac member 14, and connector 16, 16′ all operate in unison. However, certain embodiments may use less than all of these components.

Cardiac member 14 may be made of a generally resilient material having sufficient flexibility to allow it to be compacted to pass through the esophagus while having sufficient rigidity to allow it to transmit strain from connector 16 to the cardiac region of the stomach. In the illustrated embodiment, body 21 of cardiac member 14 is made from a molded silicone, such as 60 durometer LSR silicone with an embedded fabric mesh 23 of the type that is known in the art, such as a precision woven polypropylene 35.5×35.5 mesh or a Nitinol mesh. The mesh increases tear resistance and stiffness. In the illustrated embodiments, cardiac member 14 is configured to engage the cardia and not the fundus of the stomach. The cardia is resistant to dilation due to its structure while the fundus is subject to dilation. Therefore, cardiac member 14 stimulates the mechanoreceptors and reforms the cardia without causing substantial dilation.

Tension member 24 and tether(s) 26 as well as the esophageal and cardiac members may further be as disclosed in International Patent Application Publication Nos. WO 2008/101048 and WO 2008/101078, the disclosures of which are hereby incorporated herein by reference.

Changes and modifications in the specifically described embodiments can be carried out without departing from the principles of the invention which is intended to be limited only by the scope of the appended claims, as interpreted according to the principles of patent law including the doctrine of equivalents.

The embodiments of the invention in which an exclusive property or privilege is claimed are defined as follows:

1. A method of reducing a hiatal hernia, said method comprising:
positioning a device at the gastro-esophageal region of the patient, said device having a wall that is generally configured to the normal anatomy at the gastro-esophageal region of a person without a hiatal hernia; and
using said device to form the anatomy of the patient at the gastro-esophageal region substantially to the normal anatomy of the person without a hiatal hernia.

2. The method as claimed in claim 1 wherein said device includes a cardiac member having a surface that is generally configured to the normal anatomy at the cardiac region of a person without a hiatal hernia and positioning said cardiac member against the cardiac portion of the stomach to restore the shape of the cardiac portion of the stomach substantially to the shape of the cardiac portion of the stomach of the person without a hiatal hernia.

3. The method as claimed in claim 2 wherein said device includes an esophageal member having a surface that is generally configured to the normal anatomy at the distal esophagus of a person without a hiatal hernia and positioning said esophageal member within the distal portion of the distal esophagus adjacent the gastro-esophageal (GE) junction to restore the shape of the distal esophagus substantially to the shape of the distal esophagus of a normal person without a hiatal hernia.

4. The method as claimed in claim 1 wherein said device includes a connector, said connector connected with the esophageal member and the cardiac member to establish intra-abdominal relationship of the distal esophagus, the GE junction and the cardiac region of a normal person without a hiatal hernia.

5. The method as claimed in claim 4 including positioning said connector at the GE junction with a contiguous portion of the GE junction substantially unrestrained.

6. The method as claimed in claim 4 including positioning at least a portion of said connector within the pseudo-sphincter comprising the GE junction.

7. The method as claimed in claim 4 including positioning at least a portion of said connector outside of the pseudo-sphincter comprising the GE junction.

8. The method as claimed in claim 4 including affecting the angle of HIS of the patient by positioning said cardiac member with respect to said esophageal member with said connector.

9. The method as claimed in claim 8 wherein said positioning includes drawing said cardiac member toward said esophageal member.

10. The method as claimed in claim 1 wherein said positioning a device includes positioning an esophageal member having a surface that is generally configured to the normal anatomy at the distal esophagus of a person without a hiatal hernia and positioning said esophageal member within the distal portion of the esophagus adjacent the gastro-esophageal (GE) junction to restore the shape of the distal esophagus to the shape of the distal esophagus of a normal person without a hiatal hernia.

11. The method as claimed in claim 1 wherein said device does not substantially restrict passage of food.

12. The method as claimed in claim 1 wherein said using said device to form the anatomy of the patient at the gastro-esophageal region to the normal anatomy of a person without a hiatal hernia includes applying an outward force with said wall to the gastro-esophageal region.

13. A hiatal hernia reduction device, comprising:
a wall that is generally configured to the anatomy at the gastro-esophageal region of a normal person without a hiatal hernia;
wherein said wall is adapted to form the anatomy of the patient at the gastro-esophageal region substantially to the anatomy of the normal person without a hiatal hernia.

14. The hiatal hernia reduction device as claimed in claim 13 wherein said wall defines a cardiac member having a surface that is generally configured to the normal anatomy at the cardiac region of a person without a hiatal hernia and wherein said cardiac member is adapted to restore the shape of the cardiac portion of the stomach substantially to the shape of the cardiac portion of the stomach of the person without a hiatal hernia when positioned against the cardiac portion of the stomach.

15. The hiatal hernia reduction device as claimed in claim 14 wherein said wall defines an esophageal member having a surface that is generally configured to the normal anatomy at the distal esophagus of a person without a hiatal hernia and wherein said esophageal member is adapted to restore the shape of the distal esophagus substantially to the shape of the distal esophagus of a normal person without a hiatal hernia when positioned within the distal portion of the distal esophagus adjacent the gastro-esophageal (GE) junction.

16. The hiatal hernia reduction device as claimed in claim 15 including a connector, said connector connected with the esophageal member and the cardiac member to establish intra-abdominal relationship of the distal esophagus, the GE junction and the cardiac region of a normal person without a hiatal hernia.

17. The hiatal hernia reduction device as claimed in claim 13 wherein said wall defines an esophageal member having a surface that is generally configured to the normal anatomy at the distal esophagus of a person without a hiatal hernia and wherein said esophageal member is adapted to restore the shape of the distal esophagus substantially to the shape of the distal esophagus of a normal person without a hiatal hernia when positioned within the distal portion of the distal esophagus adjacent the gastro-esophageal (GE) junction.
